(19) United States
(12) Patent Application Publication
(10) Pub. No.: US 2023/0095305 A1
(43) Pub. Date: Mar. 30, 2023

(54) COMPRESSION AID

(71) Applicant: FRESENIUS MEDICAL CARE DEUTSCHLAND GMBH, Bad Homburg (DE)

(72) Inventors: Stefan Konrad MARTERSTOCK, Dettelbach (DE); Pascal KOPPERSCHMIDT, Dittelbrunn (DE); Reiner SPICKERMANN, Wasserlosen-Burghausen (DE)

(73) Assignee: FRESENIUS MEDICAL CARE DEUTSCHLAND GMBH, Bad Homburg (DE)

(21) Appl. No.: 17/908,943

(22) PCT Filed: Mar. 2, 2021
(86) PCT No.: PCT/EP2021/055096

§ 371 Date: Sep. 2, 2022

(30) Foreign Application Priority Data
Mar. 5, 2020 (DE) 10 2020 105 891.8

(51) Int. Cl. A61B 17/132

(52) U.S. Cl. CPC A61B 17/1325; A61B 2090/0807

(57) ABSTRACT

The present invention relates to a compression aid for compressing a puncture site of a patient, wherein the compression aid comprises a peripheral band whose circumference can be adjusted, with a constant force spring being arranged in the peripheral band.

The present invention relates to a compression aid for compressing a puncture site, in particular of a venous access port of a patient.

The patient is typically heparinized, i.e. blood coagulation is still negatively impaired, after a hemodialysis treatment. As a result, the venous access port has to be compressed after the needle of the dialysis machine has been disconnected.

The compression aids known from the prior art are mostly based on a simple spring force mechanism. In this process, the force on the puncture site can be set using a rotary knob. It is implemented by a screw mechanism (force-distance relationship). The disadvantage results here that the pressure exerted can be set incorrectly. It is even more disadvantageous that the compressive force exerted is influenced on a movement/muscle tension, i.e. on a change of the arm girth, which can as a consequence produce damage to the venous access port and/or is at least unpleasant with respect to comfort in wear.

Apart from this, the known compression aids are not flat and therefore only poorly fit underneath the clothing of the patient.

It is likewise known from the prior art to utilize a compressor and an air cushion for the dynamic exertion of pressure onto the puncture site. The force or pressure exerted can always be maintained in the ideal range in this manner. The implementation is, however, comparatively expensive (medical device software and hardware). A rechargeable battery is inter alia required to operate the device. Availability is therefore only ensured if the rechargeable battery is also regularly fully charged, etc. In addition, the portability of the device is a further disadvantage in comparison with the mechanical solutions.

It is thus the underlying object of the present invention to provide a compression aid that can be reliably operated and that has a simple design.

This object is achieved by a compression aid having the features of claim 1. Provision is accordingly made that the compression aid comprises a peripheral band whose circumference can be adjusted, with a constant force spring being arranged in the peripheral band. A constant force spring brings about the advantage that unlike a conventional spring it exerts or sets a constant force over a specific distance or stroke. This make it possible to reduce the likelihood that too small or too high a pressing force is exerted on the puncture site. It is sufficient for the user to set the compression aid such that the stroke or the deflection is in the range of the constant force. The invention thus presents a constant force spring that partially provides independence between the force and the distance. Possible forms of implementation are purely mechanical or also magnetic.

Provision is made in a conceivable embodiment of the compression aid that a display element is preferably located at the outer side of the constant force spring and is configured to display the deflection of the constant force spring directly or indirectly. This makes it possible for the user to determine whether the range of the constant force has already been reached or has not yet been reached or has even been exceeded. It is the intention of the display that the constant force spring can be centrally deflected so that a setting range is obtained that is as large as possible. The force applied by the spring cannot be directly influenced by this.

The advantage of the display is the optimization of the active range in which the spring can hold the force constant.

The display element is preferably configured to display the range of constant force to the user.

It is conceivable that an element is provided by means of which the exerted force can be increased over the range of the constant force applied by the constant force spring. This element serves to compress the puncture site more strongly by means of a closure to stop bleeding in the case of too great a tightening outside the ideal position (center of the constant force range) or in an emergency. The element can for this purpose, for example, partially have a conventional spring constant (elastic modulus) up to an abutment or can be designed as a kind of “safety chain”.

It can in particular be necessary in a case of emergency to exert a force on the puncture site that is above the force that can be applied ideally by the constant spring. This compression required for an emergency should, however, also be limited or even unlimited (abutment). Compression can be performed at one’s own discretion in an emergency.

Two (non-restrictive) alternatives named by way of example can be considered for this purpose that are each attached to the band in parallel and detached with respect to the compression spring itself:
1) A securing element such as a safety chain that ensures that the “tightening distance” does not exceed a critical point (max.). The force exerted is thus also bounded by restricting the tightening distance. This restriction has the purpose of preventing the force of the constant force spring from decreasing on too great a tightening (cf. characteristic curve in FIG. 2). As soon as the spring is outside the range between min. and max., the spring possibly practically no longer provides any force, i.e. the compression aid could release completely, i.e. could break apart into two parts.
2) Embodiment with spring: A further scale can optionally be provided for this purpose that displays the exerted force relative to the elastic modulus of the material used. A fine adjustment in the end range of the maximum applicable force can hereby be implemented.

The spring stiffness can be adapted relative to the characteristic curve of the constant force spring, in particular while taking account of the point P1, i.e. the ideal position.

It must be ensured for the maximum exertable force that the closure of the band does not slip out or open accidentally.

A conceivable embodiment thus comprises the element being a spring that preferably at least regionally has a linear dependency between the stroke and the force, i.e. like a conventional spring.

Said element is preferably connected in parallel with the constant force spring. A serial connection is also conceivable and covered by the invention.

A serial connection, optionally in combination with an abutment, is sensible so that on an overexpansion into the abutment of the constant force spring, the latter then pauses and the conventional spring, connected in series, then delivers a force-stroke characteristic curve above the force of the constant force spring.

The band can have two ends, with the one end of the band being insertable into the other end of the band and being fixable there, as is known from a cable tie. Other closures are also conceivable and covered by the invention.

Provision is preferably made that the band has a closure that is manually releasable (reuse, removal of the compression aid).

The constant force spring can be designed as mechanical and/or as magnetic. The term “spring” must thus have a broad interpretation and also comprises magnetic embodiments or e.g. also purely mechanical variants.

It is conceivable that the compression aid has a color coding that is correlated with the force applied by the constant force spring. Which constant force can be implemented with which compression aid is thus immediately made known to the user. This also reduces the risk of an incorrect use by confusing the available compression aids since a patient becomes aware of his level, i.e. optionally his color, over time and he can recognize if he were to receive an incorrect compression aid from staff.

The present invention also relates to a method of compressing a puncture site such as a fistula or another blood vessel, in particular the access port or ports of a dialysis patient.

The compression aid is used when the needle or canula has been removed such as after the performing of a dialysis treatment.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the invention will be explained in more detail with reference to an embodiment shown in the drawing.

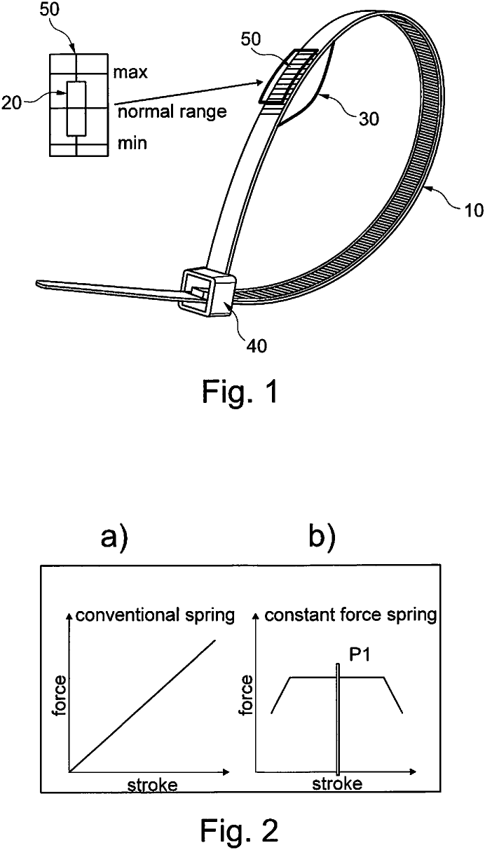

There are shown: FIG. 1: a perspective view of a compression aid in accordance with the invention; and

FIG. 2: force-distance curves of a conventional spring and of a constant force spring.

FIG. 1 shows a compression aid in accordance with the invention in a perspective view.

The compression aid comprises a peripheral band 10 in which a constant force spring 20 is integrated over a partial circumference. As can be seen from FIG. 2b, it has the advantage that the spring exerts a constant force on the puncture site over a specific stroke. It thus differs from the characteristic curve of a conventional spring in accordance with FIG. 2a in which the exerted force increases linearly with the deflection, i.e. with the stroke.

The constant force spring has the effect that the force by which the band is pressed onto the puncture site is constant over a specific stroke. The ideal position is marked by the reference numeral P1 in FIG. 2b. This position represents the center of the stroke at a constant stroke.

Reference numeral 30 characterizes an element in the form of a conventional spring that, as shown in FIG. 1, is arranged in parallel or in series with the constant force spring 10. The conventional spring is, however, not present in a preferred embodiment.

This element 30 serves to compress the puncture site more strongly by means of the closure 40 to be able to stop bleeding in the case of too great a tightening outside the ideal position P1 or in an emergency. The element 30 can for this purpose e.g. partially have a conventional spring constant (elastic modulus) up to an abutment or can be designed as a kind of “safety chain”.

As can be seen from FIG. 1, the band can be designed as a cable tie or like a cable tie. The term “band” is to be given a broad interpretation and also comprises cuffs, etc., for example.

A scale in the form of a transparent window (e.g. of plastic) is marked by reference numeral 50 and is preferably located above the constant force spring.

The scale has a max. and a min. mark that display the range of the constant force. It serves the ideal tightening of the cable tie or of another band. The normal range that is marked by P1 in FIG. 2b is located between the max. and min. marks.

The closure 40 can furthermore again be openable (reuse, removal of the compression aid).

Different strengths of the compression aid (constant force spring) can be provided, also while taking account of the girth of the forearm of a person/patient. This can e.g. be made visible by color coding of the “compression aid/bracelet”.

There can be named as advantages with respect to the prior art in a preferred embodiment:
Intrinsically safe since too tight a compression can be precluded from a construction aspect by the constant force spring to the extent that said element is not provided. If it is provided, it is, however, made clear to the operator by the scale that he is leaving the range of the mechanical compression adjustment by the constant force spring element;
The compression aid is preferably again manually openable via the closure;
Simple in design and thus inexpensive. No expensive and complex hardware and software development. Simplification of the risk analysis;
Increased comfort in wear (due to slim design without wiring) and mechanical compression regulation by a constant force spring;
Abutment for tightening in an emergency with impending blood loss possible;
Risk of damage to the venous access port drops;
Compensation of muscle movements by the arm/hand (mechanical adjustment);
Simple to use, i.e. also in the domestic area by less well-trained staff;
High availability (no rechargeable battery and no charge status dependency, ...);
Reusable or as disposable business for reasons of hygiene;
Greater confidence of the users since the risk of too great a tightening is eased or is prevented from a construction aspect;
Easier technical implementation without any great sustaining effort (discontinuation of electronics hardware, ...);
Can be implemented less expensively.

1. A compression aid for compressing a puncture site of a patient, characterized in that the compression aid comprises a peripheral band whose circumference can be adjusted, with a constant force spring being arranged in the peripheral band.

2. A compression aid in accordance with claim 1, characterized in that a display element is preferably located at the outer side of the constant force spring and is configured to display the deflection of the constant force spring.

3. A compression aid in accordance with claim 2, characterized in that the display element is configured to display the range of the constant force to the user.

4. A compression aid in accordance with claim 1, characterized in that an element is provided by means of which the exerted force can be increased over the range of the constant force applied by the constant force spring.

5. A compression aid in accordance with claim 4, characterized in that the element is a spring that preferably at least regionally has a linear dependency between the stroke and the force.

6. A compression aid in accordance with claim 4, characterized in that the element is connected in parallel with the constant force spring.

7. A compression aid in accordance with claim 1, characterized in that the band has two ends, with the one end of the band being insertable into the other end and being fixable there.

8. A compression aid in accordance with claim 1, characterized in that the band has a closure that is manually releasable.

9. An compression aid in accordance with claim 1, characterized in that constant force spring is designed as mechanical and/or magnetic.

10. A compression aid in accordance with claim 1, characterized in that the compression aid has a color coding that is correlated with the force applied by the constant force spring.
